(19) United States
(12) Patent Application Publication
(10) Pub. No.: US 2010/0055177 A1
(43) Pub. Date: Mar. 4, 2010

(54) MODIFIED RELEASE COMPOSITION OF LEVETIRACETAM AND PROCESS FOR THE PREPARATION THEREOF

(76) Inventors: Dafna Arieli, Kadima (IL); Orly Grinberg, Ganey Tikva (IL); Roey Solomonovich, Tel Aviv (IL)

Correspondence Address: KENYON & KENYON LLP, ONE BROADWAY, NEW YORK, NY, 10004, US

(21) Appl. No.: 12/549,727

(22) Filed: Aug. 28, 2009

Related U.S. Application Data
(60) Provisional application No. 61/190,560, filed on Aug. 29, 2008.
(60) Provisional application No. 61/180,980, filed on May 26, 2009.

(51) Int. Cl. A61K 9/54; A61K 9/26; A61K 9/16; A61K 31/40

(52) U.S. Cl. 424/458; 424/469; 424/490; 514/424

(57) ABSTRACT

Provided are modified release levetiracetam compositions, and processes for preparing them.

### CROSS REFERENCE

The present invention claims the benefit of the following U.S. Provisional patent application Nos. 61/190,560 filed Aug. 29, 2008; and 61/180,980 filed May 26, 2009. The contents of these applications are incorporated herein by reference.

### FIELD OF THE INVENTION

The present invention relates to modified release levetiracetam compositions, and to processes for preparing the compositions of the invention.

### BACKGROUND OF THE INVENTION

Levetiracetam is reported to be an antiepileptic drug. Apparently, only one enantiomer of levetiracetam, (−)-(S)-(alpha)-ethyl-2-oxo-1-pyrrolidine acetamide, is active. Levetiracetam is reported to be represented by the structural formula:

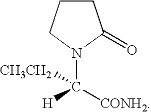

Levetiracetam is marketed by UCB Pharma, Inc. under the registered trademark KEPPRA®. KEPPRA® is available as immediate release tablets containing 250, 500, 750, or 1000 mg of the labeled compound, and as extended release tablets containing 500 and 750 mg of the labeled compound. Reportedly, the drug is rapidly and almost completely absorbed after oral administration. Apparently, levetiracetam tablets have an oral bioavailability of 100 percent, and linear pharmacokinetics over the dosage range of 500 to 5000 mg.

US 2008/0014264, US 2008/0014271, and WO 2008/006528, allegedly, describe pharmaceutical compositions in the form of a tablet comprising levetiracetam as the active ingredient and 5.0 to 59.0 percent w/w of at least one hydrophilic matrix agent as an excipient within the core.

US 2006/0165796 and US 2007/0092569, supposedly, describe an extended release tablet of levetiracetam with the core comprising levetiracetam and a water dispersible rate controlling polymer. The tablet is optionally functionally coated with a coating comprising a combination of water non-dispersible and/or water dispersible polymer.

WO 2008/062446 describes an extended release composition of levetiracetam, which, apparently, exhibits no adverse food effect, comprising from about 30 to about 85 percent w/w levetiracetam and from about 1 to 50 percent w/w of a water dispersible rate controlling polymer. WO 2009/087675, supposedly, describes a coated extended release pharmaceutical composition of levetiracetam, wherein the core is coated with a rate controlling composition.

WO 2006/123357, allegedly, relates to an oral controlled release pharmaceutical composition in the form of a unit dosage form comprising (a) levetiracetam or a pharmaceutically acceptable salt and (b) a rate controlling means comprising a rate controlling agent and/or a coating selected from an active ingredient permeable coating surrounding the unit dosage form and an active ingredient impermeable coating covering one or more surfaces, but not all surfaces of the unit dosage form. The composition is in the form of a compact tablet and the levetiracetam is present in an amount ranging from about 55 to 90 percent by weight of the tablet.

US 2009/0123541 relates to formulations described in WO 2006/123357, wherein levetiracetam or a pharmaceutically acceptable derivative thereof is, seemingly, present in an amount equivalent to from about 750 mg to about 1600 mg of levetiracetam.

WO 2006/088864 relates to a controlled release composition comprising levetiracetam for the treatment of epilepsy. Apparently, the controlled release composition comprises an immediate release component and a modified release component or formulation. According to WO'864, the modified release formulation is preferably in the form of an erodable formulation, a diffusion controlled formulation or an osmotic controlled formulation. The combination of the immediate release and the modified release components, allegedly, deliver the active in a pulsed or bimodal manner.

US 2004/0096501, apparently, relates to a modified release dosage form, comprising a high solubility active ingredient, prepared using a dual retard technique to control the release of the high solubility active ingredient, the dosage form comprising (a) micro matrix particles containing active ingredient(s) and one or more hydrophobic release controlling agent and (b) a coating of one or more hydrophobic release controlling agents on the micromatrix particles.

According to the abstract of WO 2009/069089, WO'089 relates to “a controlled release pharmaceutical composition which comprises (a) an immediate release core comprising of levetiracetam; and (b) a release rate-controlling membrane coating of hydrophobic polymer(s). There is still however a need to provide further controlled release formulations of a high solubility active drug, such as levetiracetam.”

### SUMMARY OF THE INVENTION

The present invention provides a modified release single dosage unit, comprising hydrophobic polymers as an extended release coating agent. Preferably, the hydrophobic polymers are not combined with hydrophilic pore-forming polymers.

The formulations of the invention have dissolution profiles having high stability, and can be prepared by simple procedures, according to conventional granulation and coating technologies.

In one embodiment, the invention provides a controlled release multiparticulatepharmaceutical composition, comprising levetiracetam particles coated with at least one controlled release layer. The particles may be granules comprising levetiracetam and, optionally, one or more pharmaceutically acceptable excipients. The controlled release layer may comprise at least one hydrophobic excipient, preferably a hydrophobic polymer. Preferably, except for the controlled release layer, the particles, preferably the granules do not contain a hydrophobic polymer. Said coated granules can either be compressed directly into a tablet or admixed with other excipients to form a final blend. Optionally, the final blend contains additional excipients, such as a lubricant, disintegrant, hydrophobic release controlling agent, compacting agent, or a combination thereof. These additional excipients may be extra-particular.

In a second embodiment, the invention provides a controlled release multiparticulate pharmaceutical composition, where the particles are particles of levetiracetam, in which the levetiracetam particles are coated with at least one controlled release layer. Preferably the particles comprise only levetiracetam prior to coating and preferably the controlled release layer comprises at least one hydrophobic excipient, preferably a hydrophobic polymer. Said coated levetiracetam particles can either be compressed directly into a tablet or admixed with other excipients to form a final blend. Optionally, the final blend further contains additional excipients, such as a lubricant, disintegrant, hydrophobic release controlling agent, compacting agent, or a combination thereof. These additional excipients may be extra-particular.

In a third embodiment, the invention provides a process for preparing a pharmaceutical composition. The process comprises, the steps of forming particles, preferably granules or particles of levetiracetam, or a combination thereofwhich comprise levetiracetam and, optionally, one or more pharmaceutically acceptable excipients, coating the multiparticulates with a controlled release layer, which comprises a hydrophobic excipient, preferably hydrophobic polymer, optionally blending the coated particles with one or more further pharmaceutically acceptable excipients, and compressing the resulting particles to form a tablet. A cosmetic top coat over the compressed tablet can optionally follow, preferably with a non-functional, aesthetic coating.

The present invention also provides a method for preparing a controlled release multiparticulate pharmaceutical composition comprising controlled release levetiracetam particles comprising:
a) forming granules which consist of levetiracetam and, optionally, one or more pharmaceutically acceptable excipients,
b) coating the granules with a controlled release layer,
c) optionally, blending the coated granules with one or more further pharmaceutically acceptable excipients,
d) compressing the resulting granules to form a tablet; and
e) optionally, coating the compressed tablet with a cosmetic coat.

The present invention further provides a method for preparing a controlled release multiparticulates pharmaceutical composition of levetiracetam comprising:
a) coating levetiracetam particles with a control release layer,
b) optionally, blending the coated particles with one or more pharmaceutically acceptable excipients,
c) compressing the resulting granulate/blend to form a tablet; and
d) optionally, coating the compressed tablet with a cosmetic coat.

In one embodiment of the fourth embodiment described above, the process is employed to prepare a pharmaceutical composition according to the first and second embodiments of the invention described above.

### BRIEF DESCRIPTION OF THE FIGURES

FIG. 1 illustrates the dissolution data of Tablets C and D of Example II.

FIG. 2 illustrates the dissolution data of Tablet E and F of Example III.

FIG. 3 illustrates the dissolution data of Tablet G of Example IV.

FIG. 4 illustrates the dissolution data of Tablet H, I and J of Example V.

FIG. 5 illustrates the dissolution data of Tablet K of Example VI.

FIG. 6 illustrates the dissolution data of Tablet L and O of Example VII.

FIG. 7 illustrates a tablet containing an extended release coated granulate.

### DETAILED DESCRIPTION OF THE INVENTION

The term “controlled release” is used herein according to its ordinary meaning in the art, which means a release profile with a predetermined range of release rate and for a predetermined duration of time. For example, a levetiracetam “controlled release” composition can release substantially all of the active ingredient in the composition in about 8 hours or less than about 8 hours, e.g. between about 1 hour and about 8 hours, between about 2 hours and about 7 hours, or between about 3 hours and about 6 hours when measured in-vitro. The term “substantially all of the active ingredient” means at least 80 percent by weight of the active ingredient, more preferably at least 90 percent, more preferably at least 95 percent, more preferably 100 percent, e.g. between 80 and 100 percent.

As used herein the term “compressed pharmaceutical dosage form” means a compressed (syn. compacted) pharmaceutical composition, preferably a tablet, and a “compactable agent” means a compressible agent. As also used herein, the term “unmicronized levetiracetam” means that the levetiracetam has not been micronized.

As used in the second embodiment, the term “levetiracetam particles” can be agglomerated particles, single crystals, spray dried particles, amorphous particles, etc.

The dissolution is tested using 900 ml water at 37° C. and 100 rpm using a USP dissolution test apparatus II (Paddle), or using 900 ml buffer phosphate pH 6.0 at 37° C. using USP dissolution test apparatus I (Basket). As used herein the term “stable dissolution profile” is such that upon storage of the final formulation at accelerated stability conditions (40° C./75% RH [Relative Humidity]) for a period of three months the amount of active ingredient dissolved, from the dosage, after 4 h in use medium has not changed by more than 5% of its original value (for example, the change is between 0.5% and 5%, between 1% and 4% or between 2% and 3%), wherein use medium is one of the medium applied to test dissolution as described in the application.

In one embodiment, the controlled release profile can be up to about 50% release of the active ingredient in one hour, up to about 70% in two hours, and about 80% to about 100% in about 8 hours.

Tablet hardness is a measure of the tablet's propensity to fracture under applied pressure. Devices for measuring hardness are commercially available from a variety of manufacturers such as KRAEMER (UTS) Ltd. Preferably, the hardness range of the tablets in the present invention is between about 15 to about 40 Strong-Cobb units (SCU) when measured using a KRAEMER (UTS), e.g. 15 to 30 SCU, preferably 20 to 28 SCU, more preferably 22 to 26 SCU.

In all embodiments of the present invention it is the levetiracetam particles (e.g. granules) that are individually coated with the at least one controlled release layer. One of ordinary skill of art would appreciate that in the manufacturing process, it is possible that a minimal amount of the particles (less than 1%) may not get coated due to the quality of the coating process. Preferably, each dosage form (such as tablet) has about 100 or more particles.

The at least one controlled release layer may comprise at least one hydrophobic excipient, preferably hydrophobic polymer. In all embodiments of the invention, the hydrophobic polymer may be present in an amount from about 75 to about 95 percent wt. per weight of the controlled release layer, preferably, from about 85 to about 95 percent wt. per weight of the controlled release layer, more preferably, about 85 to about 90 percent wt. per weight of the controlled release layer, and, more preferably, about 87 percent wt. per weight of the controlled release layer.

In the first embodiment of the invention, the hydrophobic polymer may be present in an amount from about 10 to about 20 percent, preferably about 15 percent wt. per total weight of the pharmaceutical composition.

In the second embodiment of the invention, the hydrophobic polymer may be present in an amount from about 15 to about 40 percent, preferably about 25 percent wt. per total weight of the pharmaceutical composition.

Examples of suitable hydrophobic excipients include, but are not limited to hydrophobic cellulose ethers, such as ethyl cellulose, cellulose acetate, polyvinyl acetate, methacrylic acid esters neutral polymer, polyvinyl alcohol-maleic anhydride copolymers, magnesium stearate, waxes, oils and the like and combinations thereof. Preferably, the hydrophobic excipient is hydrophobic polymer. More preferably, the hydrophobic polymer is ethyl-cellulose

In all embodiments of the invention, the hydrophobic polymer may be a medium to low viscosity hydrophobic polymer, such as ethyl cellulose, having a viscosity of about 7 to about 100, preferably about 7 to about 50, more preferably about 7 to about 10, most preferably about 7 cPs (centiposes). Preferably the viscosity of ethylcellulose is measured as a 5 percent w/w solution in an organic solvent of 80 percent w/w toluene and 20 percent w/w ethanol. The measurement is done using an Ubbelohde viscometer at 25° C. Such polymers provide improved control of the in vitro dissolution of a very soluble drug, such as levetiracetam (water solubility of about 1.04 g/ml). Such compositions of the invention sustain the release of levetiracetam for a period of from about 4 to about 16 hours.

In one embodiment of all aspects of the invention, the hydrophobic polymer is a film forming polymer. In a further embodiment, the hydrophobic polymer is ethyl-cellulose.

In one embodiment of all aspects of the invention, the controlled release layer is free or at least substantially free of hydrophilic pore-forming polymers

In all embodiments of the invention, the controlled release layer may further comprise a plasticizer. Plasticizers useful in the invention can include, by way of example and without limitation, hydrophobic low molecular weight polymers, hydrophobic oligomers, hydrophobic copolymers, oils, small organic molecules, ester-type plasticizers, glycol ethers, hydrophobic multi-block polymers, single block polymers, citrate ester-type plasticizers, triacetin, propylene glycol, and glycerin. Such plasticizers can also include ethylene glycol, 1,2-butylene glycol, 2,3-butylene glycol, styrene glycol, diethylene glycol, triethylene glycol, tetraethylene glycol, mono-propylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate. In addition, a combination of the plasticizers can be used in the present formulation.

In a preferred embodiment, the plasticizer is anhydrophobic plasticizer. The hydrophobic plasticizer can be selected from hydrophobic low molecular weight polymers, hydrophobic oligomers, hydrophobic copolymers, oils, small organic molecules, ester-type plasticizers such as diethyl phtalate and dibutyl sebacate, hydrophobic multi-block polymers, or combinations thereof. A preferred plasticizer is dibutyl sebacate.

In all embodiments of the invention, the plasticizer may be present in an amount from about 5 to about 25 percent wt. per weight of the controlled release layer, preferably from about 10 to about 15 percent wt. per weight of the controlled release layer, more preferably about 13 percent wt. per weight of the controlled release layer.

In all embodiments of the invention, the plasticizer may be present in an amount from about 0.5 to about 4 percent, preferably from about 1 to about 3 percent, more preferably about 2 percent wt. per total weight of the pharmaceutical composition. Preferably, in all embodiments of the present invention, the controlled release coating does not comprise a hydrophilic pore-forming polymer.

In all embodiments of the invention, the pharmaceutical composition may also contain one or more pharmaceutically acceptable excipients or adjuvants. Selection of excipients and the amount of such excipients used may be readily determined by a formulation scientist based upon experience and consideration of standard procedures and reference works in the field. The pharmaceutically acceptable excipients or adjuvants may be extra-particular and/or intra-particular (i.e. included as part of the particle).

Preferably, in all embodiments of the invention, the intra-particular excipient is at least one pharmaceutically acceptable excipient selected from the group comprising a binder, a lubricant, a disintegrant, and combinations thereof.

In all embodiments of the invention, the composition may optionally contain a binder. Preferably the binder is a non release rate controlling binder, and/or the binder is water soluble. The binder can be selected from polyvinyl pyrrolidone, low viscosity cellulose derivatives (such as hydroxypropyl cellulose, hydroxypropyl methylcellulose, hydroxyethyl cellulose and methyl cellulose), starch, pregelatinized starch, modified corn starch, polyacryl amide, poly-N-vinyl amide, sodium carboxymethyl cellulose, polyethylene glycol, gelatin, poly propylene glycol, tragacanth, alginic acid, combinations thereof and other materials known to one ordinary skill in the art. The binder, preferably when included as part of the intra-particular excipient, may be present in an amount from about 0.5 percent to about 10 percent, preferably from about 1 percent to about 5 percent, more preferably from about 3 percent to about 5 percent by weight of the pharmaceutical composition. In one embodiment, the binder is a hydrophilic binder. A preferred binder is a soluble grade polyvinyl pyrrolidone, such as PVP K-30, PVP K-90 or a combination thereof.

The binder may be included as part of the particle (e.g. granule), and/or included as an extra-particular (e.g. extra-granular) excipient in the composition.

The binder may be included as an extra-granular and/or an intra-granular excipient in any composition of the present invention. Preferably, the granules comprise levetiracetam and a binder. Preferably, the intra granular binder is a soluble grade polyvinyl pyrrolidone, such as PVP K-30, PVP K-90 or a combination thereof.

In all embodiments of the invention, the pharmaceutical composition may optionally contain a lubricant. When a dosage form, such as a tablet, is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate or combinations thereof. A preferred lubricant is magnesium stearate.

In all embodiments of the present invention, a lubricant, preferably when included as part of the extra-particular excipient, may be present in an amount from about 0.1 to about 8 percent wt. per total weight of the pharmaceutical composition, preferably from about 0.3 to about 7 percent wt. per total weight of the pharmaceutical composition, and, most preferably, from about 0.5 to about 6 percent wt. per total weight of the pharmaceutical composition.

In all embodiments of the present invention, the pharmaceutical composition may optionally contain a disintegrant. Suitable disintegrants are starch, pregelatinized starch, guar gum, low viscosity methylcellulose, and microcrystalline cellulose. A preferred disintegrant is microcrystalline cellulose.

The disintegrant may be included as part of the particle (e.g. granule), and/or included as an extra-particular (e.g. extra-granular) excipient in the composition. Preferably, the disintegrant may be included as an extra-granular excipient in the composition.

A disintegrant, preferably when included as part of the extra-particular excipient, may be present in an amount from about 1 to about 50 percent wt. per total weight of the pharmaceutical composition, preferably from about 5 to about 20 percent wt. per total weight of the pharmaceutical composition, more preferably about 10 percent wt. per total weight of the pharmaceutical composition.

In all embodiments of the present invention, the pharmaceutical composition may optionally contain a compactable excipient as an extra-particular excipient. Preferred compactable excipients are microcrystalline cellulose and dibasic calcium phosphate.

A compactable excipient may be present in an amount from about 5 to about 20 percent wt. per total weight of the pharmaceutical composition, preferably from about 7 to about 15 percent wt. per total weight of the pharmaceutical composition, more preferably about 10 percent wt. per total weight of the pharmaceutical composition.

In all embodiments of the present invention, the pharmaceutical composition may optionally contain at least one hydrophobic excipient as an extra-particular excipient. Preferably the at least one hydrophobic excipient is a hydrophobic release controlling agent. Preferably, the pharmaceutical composition contains at least two hydrophobic excipients as an extra-particular excipient. The hydrophobic excipient may be present in an amount from about 1 to about 10 percent, preferably from about 3 to about 8 percent, and, more preferably, from about 3 to about 6 percent wt. per total weight of the pharmaceutical composition.

Preferred hydrophobic release controlling agents are hydrogenated vegetable oil, hydrogenated castor oil, ethyl cellulose, and combinations thereof. The at least one hydrophobic release controlling agent, preferably when included as part of the extra-particular excipient, may be present in an amount from about 2 to about 15 percent and, preferably, from about 3 to about 8 percent by weight of the pharmaceutical composition. Preferably, the at least one hydrophobic release controlling agent is hydrogenated castor oil.

Preferred hydrophobic excipients are dibasic calcium phosphate, magnesium stearate, talc, hydrogenated vegetable oil, hydrogenated castor oil, ethyl cellulose and combinations thereof. A preferred pharmaceutical composition of any embodiment of the present invention comprises: levetiracetam, polyvinyl pyrrolidone, ethylcellulose, dibutyl sebacate, microcrystalline cellulose, hydrogenated castor oil, and magnesium stearate.

Other commonly used pharmaceutically acceptable excipients may also be used in all embodiments of the present invention. Examples of such excipients are talc, fumed silica, glyceryl monostearate, calcium stearate, kaolin, colloidal silica, gypsum, Tween 80, micronised silica, and magnesium trisilicate.

In one embodiment, the invention provides a controlled release multiparticulate pharmaceutical composition, comprising levetiracetam particles coated with at least one controlled release layer, where the particles are granules comprising levetiracetam and optionally one or more further pharmaceutically acceptable excipients, and where the controlled release layer comprises at least one hydrophobic excipient, preferably a hydrophobic polymer. Preferably, except for the coating layer, the granules do not contain a hydrophobic polymer. Said coated granules can either be compressed direct into a tablet or admixed with other excipients to form a final blend. Optionally, the final blend contains additional excipients, such as a lubricant, disintegrant, hydrophobic release controlling agent, compacting agent, or a combination thereof. Preferably, the pharmaceutical composition is a compressed pharmaceutical dosage form. Optionally, the compressed dosage form is further coated for cosmetic purposes.

In a second embodiment, the invention provides a controlled release multiparticulate pharmaceutical composition, where the particles are particles of levetiracetam, in which the levetiracetam particles are coated with at least one controlled release layer and one or more pharmaceutically acceptable excipients, where the particles comprise only levetiracetam prior to coating, the controlled release layer comprises at least one hydrophobic excipient, preferably a hydrophobic polymer, and, said coated levetiracetam particles can either be compressed direct into a tablet or admixed with other excipients to form a final blend. Optionally, the final blend contains additional excipients, such as a lubricant, disintegrant, hydrophobic release controlling agent, compacting agent and combinations thereof.

A preferred embodiment of the present invention is multiparticulate pharmaceutical composition comprising levetiracetam particulates, wherein the levetiracetam particulate comprises, for example, (i) granules that comprise levetiracetam and at least one pharmaceutically acceptable excipient, (ii) levetiracetam particles and (iii) combinations thereof. The levetiracetam particles may just contain levetiracetam. Each of the levetiracetam particulates are coated with at least one controlled release layer.

In another embodiment, the invention provides a controlled release multiparticulate pharmaceutical composition comprising levetiracetam, polyvinyl pyrrolidone, ethylcellulose, dibutyl sebacate, microcrystalline cellulose, hydrogenated castor oil, and magnesium stearate.

In all embodiments of the invention, as described above, the pharmaceutical dosage forms include, but are not limited to, the form of a tablet or mini tablets in capsule, and, more preferably a tablet. Capsules preferably contain mini tablets compositions within either a hard or soft shell. The shell may be made from gelatin, and optionally contain a plasticizer, such as glycerin and sorbitol, and an opacifying agent or colorant. Preferably, the capsule size is 1, 0 or 00 and, the mini tablet size is about 12 mm to about 3 mm.

A preferred embodiment of the present invention is a pharmaceutical composition comprising granules coated with a controlled release layer wherein the granules comprise levetiracetam and optionally one or more further pharmaceutically acceptable excipient and wherein the controlled release layer comprises a hydrophobic polymer. Preferably the granules do not contain hydrophobic polymer except for the coating layer.

A preferred embodiment of the present invention is a pharmaceutical composition comprising active particles coated with a controlled release layer and one or more pharmaceutically acceptable excipients wherein the active particles comprise only levetiracetam, and wherein the controlled release layer comprises a hydrophobic polymer, and optionally the final blend further contain additional excipients such as disintegrant and/or compacting agent.

A preferred embodiment of the present invention is a single composition e.g. tablet, comprising multiple levetiracetam particles coated with at least one controlled release layer and at least one extra-particular excipient. Preferably, the levetiracetam particles consist of levetiracetam or are granules of levetiracetam and at least one pharmaceutically excipient or combinations thereof. Preferably, the at least one intra-granular excipient is a binder. Preferably, the at least one extra-particular excipient is selected from the group comprising a binder, a lubricant, a disintegrant, a hydrophobic release controlling agent, a compacting agent and combinations thereof. Preferably, the at least one controlled release layer comprises at least one hydrophobic polymer. Preferably, the at least one controlled release layer further comprises a plasticizer.

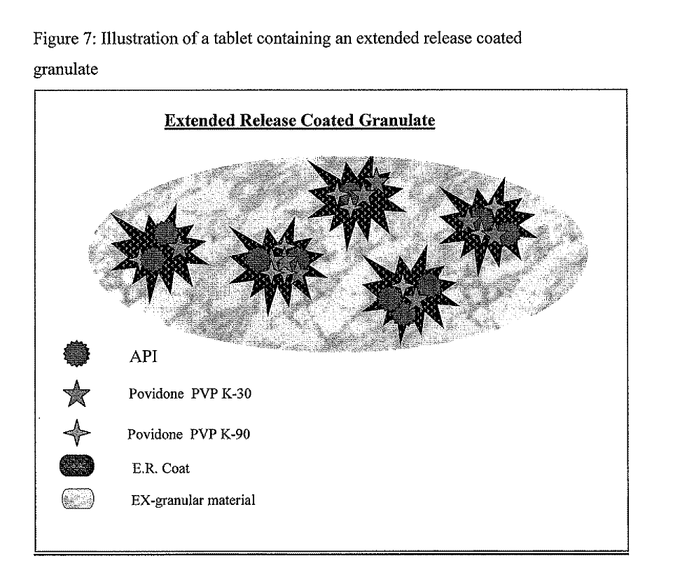

Preferably, the multiparticulate pharmaceutical compositions of the present invention are single dosage units, i.e. each single dosage unit multiparticulate composition contains greater than one levetiracetam particulate, where each particulate is coated with a controlled release layer. See, for example, FIG. 7.

Preferably, the term “multiparticulate pharmaceutical composition” is defined as encompassing a single dosage unit comprising greater than one intra-composition levetiracetam particulate, where each particulate is coated with a controlled release layer. See, for example, FIG. 7.A preferred embodiment of the present invention is a tablet comprising a compacting agent, a lubricant, a disintegrant and multiple granules comprising levetiracetam and at least one binder, wherein the granules are coated with at least one controlled release layer comprising a hydrophobic polymer and a plasticizer.

A preferred embodiment of the present invention is a tablet comprising a compacting agent, a lubricant, a disintegrant and multiple particles of levetiracetam, wherein the particles are coated with at least one controlled release layer comprising a hydrophobic polymer and a plasticizer.

A preferred embodiment of the present invention is a tablet comprising a hydrophobic release controlling agent, a compacting agent, a lubricant, a disintegrant and multiple granules comprising levetiracetam and at least one binder, wherein substantially all the granules are coated with at least one controlled release layer comprising a hydrophobic polymer and a plasticizer.

A preferred embodiment of the present invention is a tablet comprising a hydrophobic release controlling agent, a compacting agent, a lubricant, a disintegrant and multiple particles of levetiracetam, wherein substantially all the particles are coated with at least one controlled release layer comprising a hydrophobic polymer and a plasticizer.

All of the above tablets may be optionally coated with a cosmetic coat known in the art.

All of the above tablets may be produced by compressing the coated levetiracetam particles (e.g. granules) and the extra-particular excipients.

All components have the definitions as set forth above.

In one embodiment of all the above aspects of the invention, the composition sustains the release of levetiracetam for from about 4 to about 16 hours.

The present invention also provides a method for preparing a controlled release multiparticulate pharmaceutical composition comprising controlled release levetiracetam particles comprising:
a) forming granules which consist of levetiracetam and, optionally, one or more pharmaceutically acceptable excipients,
b) coating the granules with a controlled release layer,
c) blending the coated granules with one or more further pharmaceutically acceptable excipients.

The present invention further provides a method for preparing a controlled release multiparticulates pharmaceutical composition of levetiracetam comprising:
a) coating levetiracetam particles with a control release layer,
b) blending the coated particles with one or more pharmaceutically acceptable excipients.

The present invention also provides a method for preparing a controlled release multiparticulate pharmaceutical composition comprising controlled release levetiracetam particles comprising:
a) forming granules which consist of levetiracetam and, optionally, one or more pharmaceutically acceptable excipients,
b) coating the granules with a controlled release layer,
c) optionally, blending the coated granules with one or more further pharmaceutically acceptable excipients,
d) compressing the resulting granules to form a tablet; and
   optionally, coating the compressed tablet with a cosmetic coat.

The present invention further provides a method for preparing a controlled release multiparticulates pharmaceutical composition of levetiracetam comprising:
a) coating levetiracetam particles with a control release layer,
b) optionally, blending the coated particles with one or more pharmaceutically acceptable excipients,
c) compressing the resulting granulate/blend to form a tablet; and
   optionally, coating the compressed tablet with a cosmetic coat
      In one embodiment, the present invention provides a controlled release multiparticulate single dosage unit pharmaceutical composition, comprising particles of levetiracetam, the pharmaceutical composition comprises:
a) levetiracetam present in an amount from about 50 to about 80 percent, preferably from about 60 to about 70 percent wt. per total weight of the pharmaceutical composition,
b) a hydrophobic polymer present in an amount of about 10 to about 20 percent, preferably about 15 percent wt. per total weight of the pharmaceutical composition, and where the hydrophobic polymer is preferably ethyl cellulose,
c) at least one hydrophobic release controlling agent, as an extra-granular excipient, present in an amount of about 1 to about 10 percent, preferably about 5 percent wt. per total weight of the pharmaceutical composition, where the hydrophobic extra-granular excipient is hydrogenated castor oil it is preferably in an amount of at least about 4 percent wt. per total weight of the pharmaceutical composition,
d) a hydrophobic plasticizer, preferably present in an amount of about 1 to about 4 percent wt. per total weight of the pharmaceutical composition; and
e) a compactable excipient, preferably as an extra-granular excipient, preferably presents in an amount of about 5 to about 20 percent wt. per total weight of the pharmaceutical composition,
   wherein, substantially all the levetiracetam particles are coated with a controlled release layer. Preferably, the single dosage unit pharmaceutical composition is a tablet (i.e. the coated particles and the extra granular excipients are compressed to form a tablet). Preferably, the above pharmaceutical composition has a stable dissolution profile under three month accelerated conditions (40° C. and 75% RH). The granules for use in the present invention may be prepared by wet granulation. In wet granulation, some or all of the active ingredient and optionally excipients in powder form are blended (if necessary), and then further mixed in the presence of a liquid, typically water, ethanol, and/or acetone, that causes the powders to clump into granules. Part or all of the liquid may be a binder solution, such as a polyvinyl pyrrolidone solution in any of the above mentioned liquids. The granulate may be screened and/or milled, dried, and then screened and/or milled to the desired particle size. The granules are further coated with a controlled release layer, wherein the controlled release layer preferably contains a hydrophobic polymer and preferably at least one plasticizer. The granules are then dried, and may be screened and preferably further mixed with at least one pharmaceutically acceptable excipient, e.g. a binder, a lubricant, a disintegrant, a hydrophobic release controlling agent, a compacting agent and combinations thereof, prior to tableting. The granulates are then tabletted. The tablets are optionally further coated with a cosmetic coat.

The granules for use in the present invention may be prepared conventionally by dry granulation. For example, the blended composition of the active ingredient and, optionally, excipients (e.g. at least one pharmaceutically acceptable excipient selected from the group consisting of a binder, a lubricant, a disintegrant, a hydrophobic release controlling agent, a compacting agent and combinations thereof) may be compacted into a slug or a sheet, and then comminuted into compressed granules. The compacted granules may subsequently be compressed into a tablet. In one embodiment, the invention provides a process for preparing a pharmaceutical composition, comprising the steps of coating Levetiracetam particles (e.g. granules comprising levetiracetam and at least one intra-granular pharmaceutically acceptable excipient) with a control release layer, which comprises a hydrophobic polymer, optionally, blending the coated particles with one or more further pharmaceutically acceptable excipients (e.g. at least one pharmaceutically acceptable excipient selected from the group consisting of a binder, a lubricant, a disintegrant, a hydrophobic release controlling agent, a compacting agent and combinations thereof), and compressing the resulting granulate/blend to form a tablet. An additional cosmetic top coating may then be applied. The cosmetic coat may be any that is known in the art.

All components have the definitions as set forth above.

In another embodiment, the process is employed to prepare a pharmaceutical composition according to the second embodiment of the invention described above.

In one embodiment of the second embodiment of the invention, the levetiracetam particles have been purchased from Neuland Laboratories Ltd. Preferably, the levetiracetam has a median particle size of from about 200 to about 1000 microns, and, more preferably, from about 300 to about 900 microns.

In one embodiment of the second embodiment of the invention, the levetiracetam employed in the composition is unmicronized levetiracetam.

In all embodiments of the invention described herein, the controlled release layer may be applied to the granules or active particles by any conventional method. Preferably, the extended release layer is applied using a solution of at least one polymer and at least one plasticizer. Preferably, when the hydrophobic polymer is used, the coating process may be performed using a fluidized bed coater, applying the coating with the top spray method or by powder layering when waxes or oils are used.

### EXAMPLES

### Example I (Comparative)

### Extended Release Coated Tablets Produced at Large Scale Preparation of Tablets A and B

Levetiracetam (d(0.9) about 50 μm), and polyvinyl pyrrolidone (PVP K-90) were mixed into a dry blend with a high shear mixer for two minutes. The blend was further granulated with gradual addition of ethanol (95 percent) applying high motor speeds. The granules were then dried in a fluid bed dryer at about 60° C. for about 20 minutes, till the moisture content was less than about 1.5 percent. These dried granules were then milled in a Frewitt oscillating mill (1 mm followed by 0.8 mm screen), and mixed with magnesium stearate. The resulting particles were then compressed into tablets applying a tablet press machine, and coated with a controlled release layer containing ethylcellulose (ETHOCEL 7 cPs) and dibutyl sebacate employing a drum coater.

The control release coating solution was prepared by first mixing ethylcellulose in a solvent mixture of ethanol 95 percent and acetone for about 30 minutes, followed by the addition of dibutyl sebacate and polyethylene glycol and mixing for about 5 minutes. This solution was then mixed with purified water for 15 minutes to provide a homogeneous solution. Talc was mixed in a separate container with ethanol 95 percent for about 5 minutes and then the talc dispersion was sieved and mixed with the ethylcellulose containing solution for 15 minutes. The coating was performed at a nominal inlet air temperature of about 45° C. and a nominal exhaust air temperature of about 35° C., keeping the coating dispersion mixed all through the coating process. Table I summarizes the composition of the final coated tablets.

TABLE 1
Composition of tablets A & B

| | | mg/tablet | |
|---|---|---|---|
| Material | Function | A | B |
| Wet granulation | | | |
| Levetiracetam (d (0.9) ca 50 um) | API | 500.0 | 500.0 |
| Polyvinylpyrrolidone (PVP K- | Hydrophilic binder | 25.0 | 25.0 |
| 90) | | | |
| Ethanol 95%* | Granulation liquid | | |
| Ex-granular | | | |
| Magnesium Stearate | Lubricant | 5.0 | 5.0 |
| Control Release Layer | | | |
| Ethylcellulose | Hydrophobic (film | 23.1 | 15.5 |
| (ETHOCEL Premium 7 cps) | forming) polymer | | |
| Dibutyl sebacate | Hydrophobic | 1.3 | 3.5 |
| | plasticizer | | |
| Polyethylene Glycol 400 | Hydrophobic | 2.1 | |
| | plasticizer | | |
| Talc | Anti-tacking agent | | 7.7 |
| Acetone* | Process solvent | | |
| Ethanol 95%* | Process solvent | | |
| Purified Water* | Process solvent, | | |
| | plasticizer | | |
| TOTAL | | 556.5 | 556.7 |

*Removed during process

Examples of the high variability (displayed in terms of the relative standard deviation—R.S.D.) between individual tablets are provided Tables 2 and 3.

TABLE 2
Dissolution data of tablet A

| Time | Vessel | Vessel | Vessel | Vessel | Vessel | Vessel | R.S.D. |
|---|---|---|---|---|---|---|---|
| (Hrs) | 1 | 2 | 3 | 4 | 5 | 6 | (%) |
| 1 | 3 | 4 | 3 | 3 | 8 | 3 | 54.1 |
| 2 | 7 | 9 | 6 | 7 | 14 | 6 | 40.0 |
| 4 | 18 | 19 | 12 | 16 | 27 | 15 | 29.1 |
| 8 | 37 | 38 | 27 | 33 | 49 | 32 | 20.9 |
| 12 | 53 | 53 | 41 | 47 | 100 | 47 | 37.9 |

TABLE 3
Dissolution data of tablet B

| Time | Vessel | Vessel | Vessel | Vessel | Vessel | Vessel | R.S.D. |
|---|---|---|---|---|---|---|---|
| (Hrs) | 1 | 2 | 3 | 4 | 5 | 6 | (%) |
| 1 | 1 | 1 | 0 | 1 | 1 | 0 | 37.8 |
| 2 | 2 | 2 | 1 | 2 | 2 | 1 | 36.4 |
| 4 | 6 | 6 | 2 | 5 | 6 | 2 | 39.8 |
| 8 | 18 | 19 | 8 | 17 | 19 | 7 | 38.7 |
| 12 | 30 | 32 | 16 | 29 | 33 | 13 | 34.8 |

The dissolution profiles of tablets A and B with 5 percent extended release coating, are associated with high RSD, compared to tablets H, I and J, although different coating compositions are involved. This implies that the problem is not formulation dependant but rather is inherent to the overall concept.

### Example II

### Coated Granules Compressed into Tablets

Preparation of tablets C and D:

Levetiracetam (d(0.9) about 50 μm) and polyvinyl pyrrolidone (PVP K-90) were mixed into a dry blend with a high shear mixer for eight minutes. The blend was initially granulated with an ethanolic binder solution of polyvinyl pyrrolidone (PVP K-90), and then granulated further with ethanol applying high motor speeds. The granules were dried in a fluid bed dryer at about 50° C. for about 20 minutes, till the moisture content was less than about 1.5 percent. These dried granules were then milled in a Frewitt oscillating mill (1.2 mm screen). The resulting milled particles were then coated with a controlled release layer containing ethylcellulose (ETHOCEL 7 cPs) and dibutyl sebacate employing a Glatt-Powder-Coater-Granulator (GPCG).

The control release coating solution was prepared by first mixing ethylcellulose in a solvent mixture of ethanol 95 percent and acetone for about 30 minutes, followed by the addition of dibutyl sebacate and mixing for about 5 minutes. This solution was then mixed with purified water for 15 minutes to provide a homogeneous solution. The coating was performed at a nominal inlet air temperature of about 55° C. and a nominal exhaust air temperature of about 30° C. The resulting coated granules were then mixed with magnesium stearate (Tablet C) and microcrystalline cellulose (Tablet D) and compressed into tablets applying a tablet press machine.

Table 4 summarizes the composition of the final tablets.

TABLE 4
Composition of tablets C & D

| | | mg/tablet | |
|---|---|---|---|
| Material | Function | C | D |
| Wet granulation | | | |
| Levetiracetam (d (0.9) ca 50 um) | API | 500.0 | 500.0 |
| Polyvinylpyrrolidone (PVP K- | Hydrophilic | 15.0 | 15.0 |
| 90) | Binder | | |
| Binder Solution | | | |
| Polyvinylpyrrolidone (PVP K- | Hydrophilic | 10.0 | 10.0 |
| 90) | Binder | | |
| Ethanol 95%* | Granulation | | |
| | solvent | | |
| Additional Granulation Liquid | | | |
| Ethanol 95%* | Granulation | | |
| | liquid | | |
| Control Release Layer | | | |
| Ethylcellulose | Hydrophobic | 114.2 | 114.2 |
| (ETHOCEL premium 7 cps) | (film forming) | | |
| | Polymer | | |
| Dibutyl sebacate | Hydrophobic | 17.0 | 17.0 |
| | plasticizer | | |
| Acetone* | Process solvent | | |
| Ethanol 95%* | Process solvent | | |
| Purified Water* | Process solvent | | |
| Ex-granular | | | |
| Microcrystalline cellulose | Compacting | | 118.0 |
| (AVICEL PH 102) | agent, | | |
| | disintegrant | | |
| Magnesium stearate | Lubricant | 5.8 | 5.8 |
| TOTAL | | 662.0 | 780.0 |

*Removed during process

Dissolution Method and Dissolution Profile

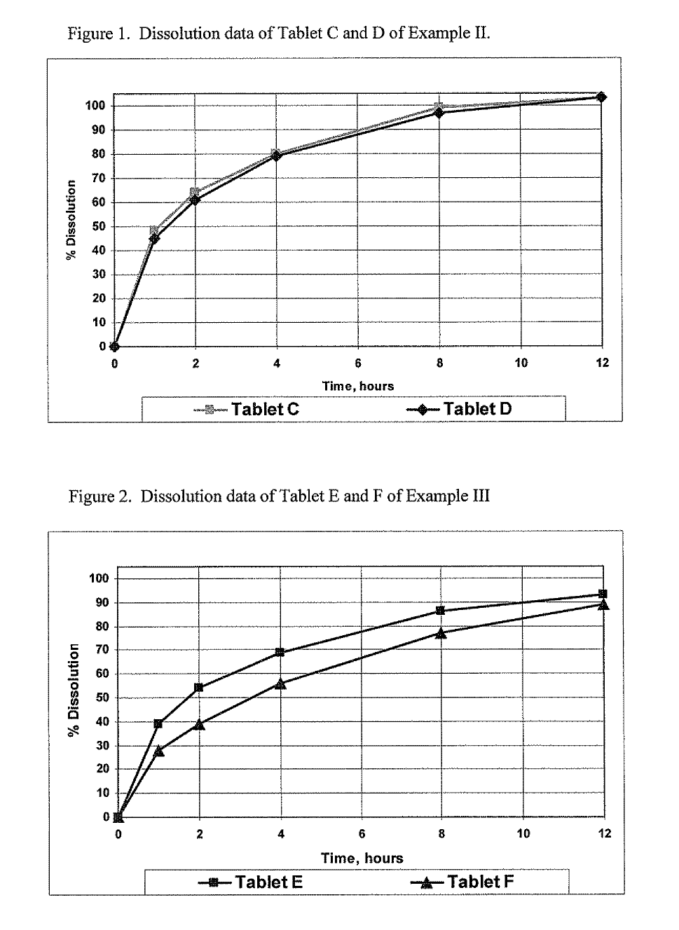

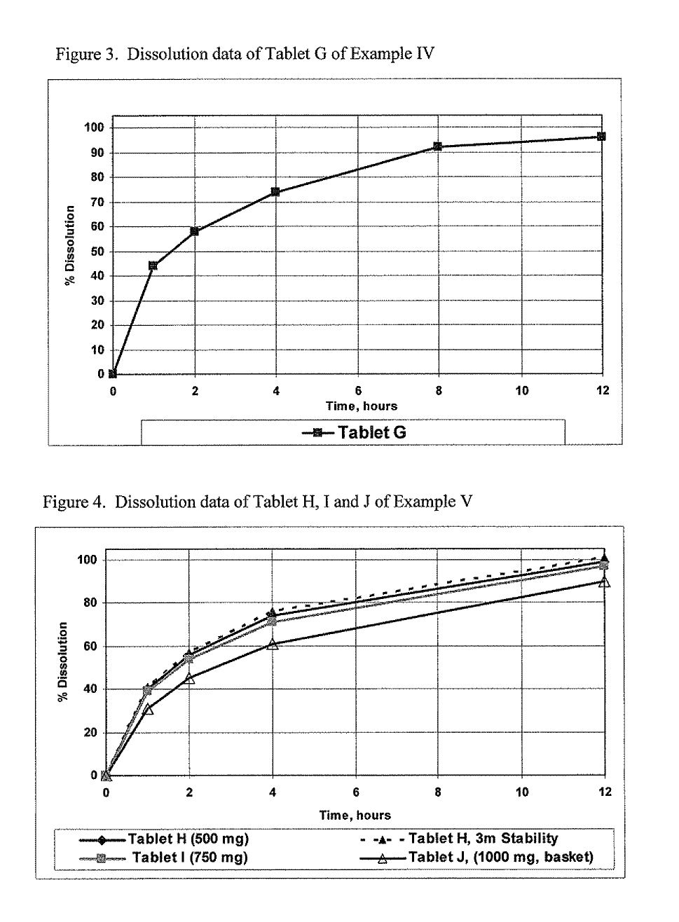

Tablets of formulations C and D were dissolved in 900 ml water at 37° C. and 100 rpm using a USP dissolution test apparatus II (Paddle). The drug release profile was measured at time intervals of 1, 2, 4, 8, and 12 hours. Table 5 and FIG. 1 summarize the dissolution data of Examples II.

TABLE 5
Dissolution Profile of Example II (%)

| Time (Hrs) | Tablet C | Tablet D |
|---|---|---|
| 1 | 48 | 45 |
| 2 | 64 | 61 |
| 4 | 80 | 79 |
| 8 | 99 | 97 |
| 12 | 103 | 103 |

### Example III

### Coated Active Particles with Ex-Granular Polymer or Disintegrant

Preparation of tablets E and F

Levetiracetam particles (d(1)=880 μm, d(0.9)=640 μm and d(0.5)=315 μm) were coated with a controlled release layer containing ethylcellulose (Ethocel 7 cPs) and dibutyl sebacate employing a Glatt-powder-coater-granulator (GPCG).

The control release coating solution was prepared by first mixing ethylcellulose in a solvent mixture of ethanol 95 percent and acetone for about 30 minutes, followed by the addition of dibutyl sebacate, and mixing for about 5 minutes. This solution was then mixed with purified water for 15 minutes to provide a homogeneous solution. The coating was performed at a nominal inlet air temperature of about 60° C. and a nominal exhaust air temperature of about 26° C. The resulting coated particles were mixed with magnesium stearate and ethylcellulose (ETHOCEL T10, Tablet E) or microcrystalline cellulose (Avicel PH 102, Tablet F) and compressed into tablets applying a tablet press.

Table 6 summarizes the composition of the final tablets.

TABLE 6
Composition of tablets E & F

| | | mg/tablet | |
|---|---|---|---|
| Material | Function | E | F |
| Core particles | | | |
| Levetiracetam (d (0.9) | API | 500.0 | 500.0 |
| ca 600 um) | | | |
| Control Release Layer | | | |
| ETHOCEL premium 7 cps | Hydrophobic (film | 87.0 | 174.0 |
| (ethylcellulose) | forming) Polymer | | |
| Dibutyl sebacate | Hydrophobic | 13.0 | 26.0 |
| | plasticizer | | |
| Acetone* | Process solvent | | |
| Ethanol 95%* | Process solvent | | |
| Purified Water* | Process solvent | | |
| Ex-granular | | | |
| Magnesium stearate | Lubricant | 6.0 | 5.0 |
| ETHOCEL T10 pharm | Hydrophobic | 74.0 | |
| (ethylcellulose) | compactable | | |
| | Polymer | | |
| Microcrystalline cellulose | Disintegrant | | 125.0 |
| (AVICEL PH 102) | | | |
| TOTAL | | 680.0 | 830.0 |

Dissolution Method and Dissolution Profile

Tablets of formulations E and F were dissolved in 900 ml water at 37° C. and 100 rpm using a USP dissolution test apparatus I (Paddle). The drug release profile was measured at time intervals of 1, 2, 4, 8, and 12 hours. Table 7 and FIG. 2 summarize the dissolution data of Examples III.

TABLE 7
Dissolution Profile of Example III (%)

| Time (Hrs) | Tablet E | Tablet F |
|---|---|---|
| 1 | 39 | 28 |
| 2 | 54 | 39 |
| 4 | 69 | 56 |
| 8 | 86 | 77 |
| 12 | 93 | 89 |

### Example IV

### Coated Active Particles with Ex-Granular Compacting Agent

Preparation of Tablet G:

Levetiracetam particles (d(1)=880 μm, d(0.9)=640 μm and d(0.5)=315 μm) were coated with a controlled release layer containing ethylcellulose (ETHOCEL 7 cPs) and dibutyl sebacate employing a glatt-powder-coater-granulator (GPCG).

The control release coating solution was prepared by first mixing ethylcellulose in a solvent mixture of ethanol 95 percent and acetone for about 30 minutes, followed by the addition of dibutyl sebacate and mixing for about 5 minutes. This solution was then mixed with purified water for 15 minutes to provide a homogeneous solution. The coating was performed at a nominal inlet air temperature of about 60° C. and a nominal exhaust air temperature of about 26° C. The resulting coated particles were mixed with magnesium stearate and calcium dibasic phosphate (anhydrous) and compressed into tablets applying a tablet press machine.

TABLE 8
Composition of tablet G

| | | mg/tablet |
|---|---|---|
| Material | Function | I |
| Core particles | | |
| Levetiracetam (d (0.9) | API | 500.0 |
| ca 600 um) | | |
| Control Release Layer | | |
| ETHOCEL premium 7 cps | Hydrophobic (film | 87.0 |
| (ethylcellulose) | forming) polymer | |
| Dibutyl sebacate | Hydrophobic | 13.0 |
| | plasticizer | |
| Acetone* | Process solvent | |
| Ethanol 95%* | Process solvent | |
| Purified water* | Process solvent | |
| Ex-granular | | |
| Magnesium stearate | Lubricant | 6.0 |
| Calcium dibasic phosphate | Compacting Agent | 74.0 |
| TOTAL | | 680.0 |

Dissolution Method and Dissolution Profile

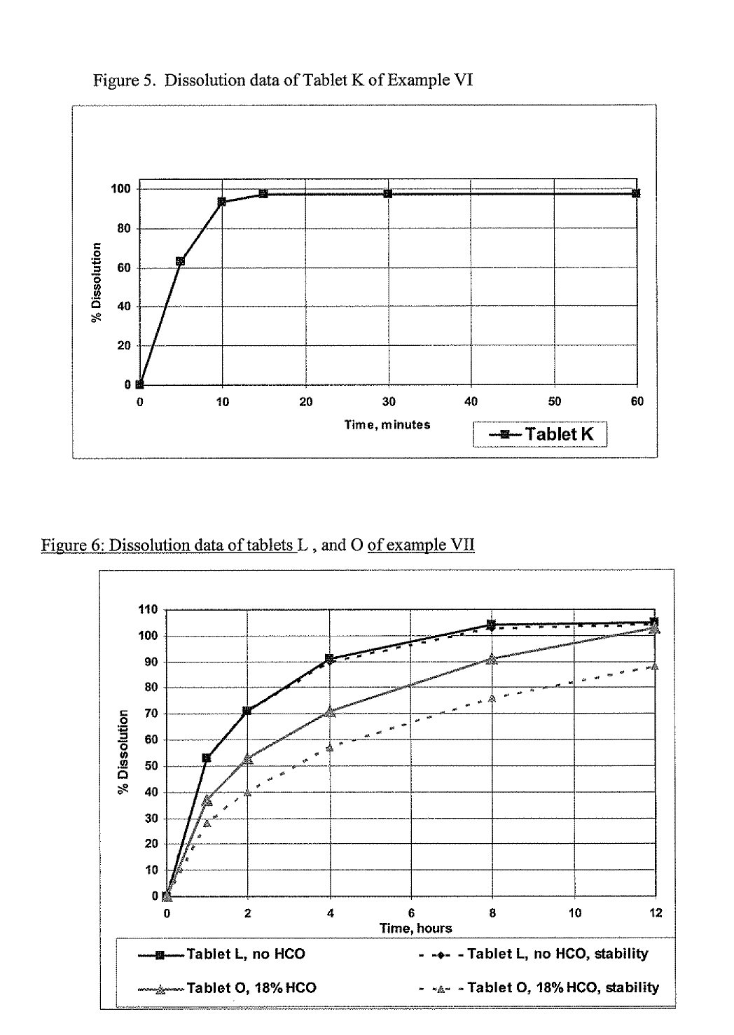

Tablets of formulation G were dissolved in 900 ml water at 37° C., 100 rpm using a USP dissolution test apparatus II (Paddle). The drug release profile was measured at time intervals of 1, 2, 4, 8 and 12 hours. Table 9 and FIG. 5 summarize the dissolution data of Example IV.

TABLE 9
Dissolution Profile of Example IV (%)

| Time (Hrs) | Tablet G |
|---|---|
| 1 | 44 |
| 2 | 58 |
| 4 | 74 |
| 8 | 92 |
| 12 | 96 |

### Example V

### Coated Granules Compressed into Tablets

Preparation of Tablets H, I and J:

Levetiracetam (d(0.9) about 50 μm), and polyvinyl pyrrolidone (PVP K-30 and PVP K-90) were mixed into a dry blend with a high shear mixer for five minutes. The blend was initially granulated with an ethanolic binder solution of polyvinyl pyrrolidone (PVP K-90) in ethanol (95 percent), then granulated with ethanol (95 percent), and finally granulated further with additional ethanol applying high motor speeds. The wet granules were milled on a Quadro Comil mill, and then dried in a fluid bed dryer at about 50° C. for about 20 minutes, till the moisture content was less than about 1.0 percent. The dried granules were then milled in a Frewitt oscillating mill (1.25 mm screen), and then coated with a controlled release layer containing ethylcellulose (ETHOCEL 7 cPs) and dibutyl sebacate employing a Glatt-powder-coater-granulator (GPCG).

The control release coating solution was prepared by first mixing ethylcellulose in a solvent mixture of ethanol 95 percent and acetone for about 30 minutes, followed by the addition of dibutyl sebacate and mixing for about 5 minutes. This solution was then mixed with purified water for 15 minutes to provide a homogeneous solution. The coating was performed at a nominal inlet air temperature of about 50° C. and a nominal exhaust air temperature of about 30° C. The resulting coated granules were mixed with microcrystalline cellulose, hydrogenated castor oil and magnesium stearate and compressed into tablets applying a tablet press machine. The compressed tablets were further coated with cosmetic coating comprising OPADRY® applying a pan coater.

Table 10 summarizes the composition of the final tablets.

TABLE 10
Composition of tablets H, I and J

| | | mg/tablet | | |
|---|---|---|---|---|
| Material | Function | H | I | J |
| Wet granulation | | | | |
| Levetiracetam (d (0.9) ca | API | 500.0 | 750.0 | 1000.0 |
| 50 um) | | | | |
| Polyvinylpyrrolidone | Hydrophilic | 3.0 | 4.5 | 6.0 |
| (PVP K-90) | binder | | | |
| Polyvinylpyrrolidone | Hydrophilic | 12.0 | 18.0 | 24.0 |
| (PVP K-30) | binder | | | |
| Ethanol 95%* | Granulation | | | |
| | liquid | | | |
| Binder Solution | | | | |
| Polyvinylpyrrolidone | Hydrophilic | 10.0 | 15.0 | 20.0 |
| (PVP K-90) | binder | | | |
| Ethanol 95%* | Granulation | | | |
| | solvent | | | |
| Additional Granulation Liquid | | | | |
| Ethanol 95%* | Granulation | | | |
| | liquid | | | |
| Control Release Layer | | | | |
| Ethylcellulose (ETHOCEL | Hydrophobic | 100.0 | 150.0 | 200.0 |
| Premium 7 cps) | (film forming) | | | |
| | polymer | | | |
| Dibutyl sebacate | Hydrophobic | 15.0 | 22.5 | 30.0 |
| | plasticizer | | | |
| Acetone* | Process solvent | | | |
| Ethanol 95%* | Process solvent | | | |
| Purified Water* | Process solvent | | | |
| Ex-granular | | | | |
| Microcrystalline cellulose | Disintegrant, | 80.0 | 120.0 | 160.0 |
| (AVICEL PH 102) | compacting | | | |
| | agent | | | |
| Hydrogenated castor oil | Hydrophobic | 40.0 | 60.0 | 80.0 |
| | release | | | |
| | controlling | | | |
| | agent, | | | |
| | Lubricant | | | |
| Magnesium Stearate | Lubricant | 6.0 | 9.0 | 12.0 |
| Top Coating | | | | |
| OPADRY 03F18435 | Cosmetic coat | 16.0 | 24.0 | 32.0 |
| Purified Water* | | | | |
| TOTAL | | 782.0 | 1173.0 | 1564.0 |
| Hardness range (SCU) | | 15-30 | 20-35 | 25-40 |

*Removed during process

### Stability Test

Tablets H were exposed to a temperature of 40° C. and 75 percent relative humidity for 3 months. The corresponding dissolution profiles showed high stability; the difference between the dissolution at all time intervals was not more than 5 percent.

### Dissolution Method and Dissolution Profile

Tablets of formulations H and I were dissolved in 900 ml water at 37° C. using a USP dissolution test apparatus II. The drug release profile was measured at time intervals of 1, 2, 4, and 12 hours. Tablets of formulation J were dissolved in 900 ml buffer phosphate pH 6.0 at 37° C. using USP dissolution test apparatus I. The drug release profile was measured at time intervals of 1, 2, 4, and 12 hours. Table 11 and FIG. 6 summarize the dissolution data (average) of Example V including dissolution stability of Tablet H. Dissolution data of six tablets from tablets H, I and J is provided in tables 12, 13 and 14.

TABLE 11
Dissolution Profile of Example V (%)

| | | Tablet H, | | |
|---|---|---|---|---|
| Time | | 3 month | | |
| (Hrs) | Tablet H | stability | Tablet I | Tablet J |
| 1 | 40 | 41 | 39 | 31 |
| 2 | 56 | 57 | 54 | 45 |
| 4 | 74 | 76 | 71 | 61 |
| 12 | 99 | 101 | 97 | 90 |

TABLE 12
Dissolution data of tablet H

| Time | Vessel | Vessel | Vessel | Vessel | Vessel | Vessel | |
|---|---|---|---|---|---|---|---|
| (Hrs) | 1 | 2 | 3 | 4 | 5 | 6 | R.S.D. |
| 1 | 39% | 40% | 40% | 40% | 41% | 35% | 5.5% |
| 2 | 55% | 56% | 56% | 56% | 57% | 53% | 2.5% |
| 4 | 73% | 75% | 74% | 74% | 76% | 73% | 1.6% |
| 12 | 98% | 100% | 98% | 99% | 100% | 95% | 1.9% |

TABLE 13
Dissolution data of tablet I

| Time | Vessel | Vessel | Vessel | Vessel | Vessel | Vessel | |
|---|---|---|---|---|---|---|---|
| (Hrs) | 1 | 2 | 3 | 4 | 5 | 6 | R.S.D. |
| 1 | 39% | 38% | 39% | 39% | 38% | 39% | 1.3% |
| 2 | 54% | 53% | 53% | 54% | 52% | 55% | 2.0% |
| 4 | 72% | 70% | 70% | 71% | 69% | 72% | 1.7% |
| 12 | 99% | 96% | 96% | 98% | 95% | 98% | 1.6% |

TABLE 14
Dissolution data of tablet J

| Time | Vessel | Vessel | Vessel | Vessel | Vessel | Vessel | |
|---|---|---|---|---|---|---|---|
| (Hrs) | 1 | 2 | 3 | 4 | 5 | 6 | R.S.D. |
| 1 | 32% | 32% | 32% | 31% | 31% | 31% | 1.7% |
| 2 | 45% | 46% | 46% | 44% | 45% | 44% | 2.0% |
| 4 | 62% | 62% | 62% | 61% | 61% | 60% | 1.3% |
| 8 | 80% | 80% | 81% | 80% | 80% | 79% | 0.8% |
| 12 | 90% | 90% | 91% | 90% | 90% | 89% | 0.7% |

### Example (Comparative) VI

### Manufacturing of Tablets Composed of Levetiracetam and PVP K-90

Preparation of Tablet K:

Levetiracetam (d(0.9) about 50 μm) and polyvinyl pyrrolidone (PVP K-90) were mixed into a dry blend with a high shear mixer for two minutes. The blend was further granulated with gradual addition of ethanol (95 percent) applying high motor speeds. The granules were dried in a Fluid Bed Dryer at about 60° C. for about 5 minutes, until the moisture content was less than about 1.5 percent. These dried granules were then milled in a Frewitt oscillating mill (0.8 mm screen), and mixed with magnesium stearate. The resulting particles were then compressed into tablets applying a single-punch tablet press.

TABLE 15
Composition of tablet K

| | | mg/tablet |
|---|---|---|
| Material | Function | K |
| Wet granulation | | |
| Levetiracetam (d (0.9) ca | API | 500.0 |
| 50 um) | | |
| Polyvinylpyrrolidone | Hydroplilic Binder | 24.0 |
| (PVP K-90) | | |
| Ethanol 95%* | Granulation liquid | |
| Ex-granular | | |
| Magnesium stearate | Lubricant | 5.3 |
| TOTAL | | 529.3 |

*Removed during process

Dissolution Method and Dissolution Profile

The tablets of formulation K were dissolved in 900 ml water at 37° C., 100 rpm using a USP dissolution test apparatus II (paddle). The drug release profile was measured at time intervals of 5, 10, 15, 30, 45 and 60 minutes. Table 16 and FIG. 7 summarize the dissolution data of Tablet K.

TABLE 16
Dissolution Profile of tablet K

| Time (Min) | % dissolved |
|---|---|
| 5 | 63 |
| 10 | 93 |
| 15 | 97 |
| 30 | 97 |
| 45 | 97 |
| 60 | 97 |

### Example VII

Tablets containing 15 percent extended release coated granulate and different amounts of Microcrystalline Cellulose and Hydrogenated Castor Oil powder. Tablets L, M, N and O were prepared according to the process described in example V.

TABLE 17
Compositions of tablets L, M, N and O

| | Tablet L | Tablet M | Tablet N | Tablet O |
|---|---|---|---|---|
| Composition | (mg/tablet) | (mg/tablet) | (mg/tablet) | (mg/tablet) |
| 15% extended | 603.7 | 603.7 | 603.7 | 603.7 |
| release coated | | | | |
| granules | | | | |
| Micro- | 111.3 | — | 76.9 | — |
| crystalline | | | | |
| Cellulose | | | | |
| (AVICEL) | | | | |
| Hydrogenated | — | — | 68.4 | 135.3 |
| Castor Oil | | | | |
| powder | | | | |
| Magnesium | 5 | 5.3 | 5 | 5 |
| Stearate | | | | |
| Tablet weight | 720 | 609 | 754 | 744 |

Dissolution results

| | | | | |
|---|---|---|---|---|
| 1 hour | 53% | 59% | 46% | 37% |
| 2 hours | 71% | 77% | 64% | 53% |
| 4 hours | 91% | 96% | 84% | 71% |
| 8 hours | 104% | 101% | 102% | 91% |
| 12 hours | 105% | 101% | 105% | 103% |

/*/**The addition of extra-granular Hydrogenated Castor oil (18 percent), as a release controlling agent, provided a lower dissolution profile stability when compared to tablets containing the same extended release coated granules, but without the Hydrogenated Castor oil, as can be inferred from FIG. 8 in which, a decrease in the dissolution rate was observed after three days of stress conditions (55° C./high humidity).

### Example VIII

### Tablets Containing Different Thickness of Extended Release Coat

Tablets P, Q and R were prepared according to the process described in example V. By increasing the amount of hydrophobic polymer, e.g. ethyl cellulose, a thicker extended release coat was created. The results as shown in Table 18 indicate the increase of the extended release coating percentage from 15 to 25 percent lead to a slower dissolution profile.

TABLE 18
Compositions and dissolution profiles of tablets P, Q and R

| | Tablet P | Tablet Q | Tablet R |
|---|---|---|---|
| Composition | (mg/tablet) | (mg/tablet) | (mg/tablet) |
| 25% extended release | 656.2 | 656.2 | 656.2 |
| coated granules | | | |
| Microcrystalline | — | 118 | 80 |
| Cellulose (AVICEL) | | | |
| Hydrogenated Castor Oil | — | — | 68 |
| powder | | | |
| Magnesium Stearate | 6 | 6 | 6 |
| Total | 662 | 780 | 810 |

Dissolution results

| | | | |
|---|---|---|---|
| 1 hour | 48% | 45% | 36% |
| 2 hours | 63% | 61% | 51% |
| 4 hours | 80% | 79% | 69% |
| 8 hours | 99% | 97% | 90% |
| 12 hours | 103% | 103% | 100% |

Dissolution Conditions and Assay Determination for all Examples Except Example J.

Equipment: USP 6-vessels, Apparatus II (paddle)

Medium: Water

Temperature: 37° C.±0.5° C.

Volume: 900 mL

Rotation speed: 100 rpm

Sampling time: 1, 2, 4, (8) and 12 hours

Method: HPLC, UV at 230 nm

Dissolution Conditions and Assay Determination for Example J:

Equipment: USP 6-vessels, Apparatus I (basket)

Medium: 0.05M Phosphate buffer pH 6.0

Temperature: 37° C.±0.5° C.

Volume: 900 mL

Rotation speed: 100 rpm

Sampling time: 1, 2, 4, 8 and 12 hours

Method: HPLC, UV at 230 nm

Column & Packing: Discovery C18, 5 μm, 50×4.6 mm, (Supelco)

Column Temperature: 30° C.

Mobile Phase Water: Acetonitrile 90:10 (v/v)

Flow Rate: 0.9 mL/min.

Detector: UV at 230 nm, 10 mm flow cell path length

Injection Volume: 10 μL

Diluent/Blank Solution: 0.05M phosphate buffer pH=6.0

Injector wash solution: Water: Acetonitrile 95:5 (v/v)

Mobile phase proportions and flow rate and column temperature may be altered in order to achieve the system suitability requirements.

1. A controlled release multiparticulate pharmaceutical composition comprising levetiracetam particles each coated with at least one controlled release layer.

2. The pharmaceutical composition of claim 1, wherein the particles are granules.

3. The pharmaceutical composition of claim 1, wherein the particles are particles of levetiracetam active ingredient.

4. The pharmaceutical composition of claim 3, wherein the particles of levetiracetam active ingredient are comprised of agglomerates.

5. The pharmaceutical composition of claim 3, wherein the particles comprise only levetiracetam prior to coating.

6. The pharmaceutical composition of claim 2, wherein the granules comprise levetiracetam and at least one pharmaceutically acceptable excipient.

7. The pharmaceutical composition of claim 6, wherein the at least one pharmaceutically acceptable excipient is selected from the group comprising a binder, a lubricant, a disintegrant, and combinations thereof.

8. The pharmaceutical composition of claim 1, wherein the controlled release layer comprises at least one hydrophobic excipient.

9. The pharmaceutical composition of claim 8, wherein the hydrophobic excipient is a hydrophobic polymer.

10. The pharmaceutical composition of claim 1, wherein the pharmaceutical composition further comprises at least one extra-particular excipient selected from the group comprising a binder, a lubricant, a disintegrant, a hydrophobic release controlling agent, a compacting agent and combinations thereof.

11. The pharmaceutical composition of claim, wherein the pharmaceutical composition is a compressed pharmaceutical dosage form.

12. The pharmaceutical composition of claim 11, wherein the pharmaceutical composition is in a form of a tablet or mini-tablets/pellets in a capsule.

13. The pharmaceutical composition of claim 9, wherein the hydrophobic polymer is present in an amount from about 10 to about 40 percent wt. per total weight of the pharmaceutical composition.

14. The pharmaceutical composition of claim 8, wherein the hydrophobic excipient is selected from the group comprising hydrophobic cellulose ethers, such as ethyl cellulose, cellulose acetate, polyvinyl acetate, methacrylic acid esters neutral polymer, polyvinyl alcohol-maleic anhydride copolymers, magnesium stearate, waxes, oils.

15. The pharmaceutical composition of claim 9, wherein the hydrophobic polymer is ethyl cellulose.

16. The pharmaceutical composition of claim 1, wherein the particles do not contain a hydrophobic polymer prior to coating.

17. The pharmaceutical composition of claim 1, wherein the controlled release layer comprises a hydrophobic plasticizer.

18. The pharmaceutical composition of claim 17, wherein the plasticizer is selected from the group comprising hydrophobic low molecular weight polymers, hydrophobic oligomers, hydrophobic copolymers, oils, small organic molecules, ester-type plasticizers such as diethyl phtalate and dibutyl sebacate, hydrophobic multi-block polymers or combinations thereof.

19. The pharmaceutical composition of claim 17, wherein the plasticizer is dibutyl sebacate.

20. The pharmaceutical composition of claim 17, wherein the plasticizer is present in an amount from about 5 to about 25 percent wt. per total weight of the pharmaceutical composition.

21. The pharmaceutical composition of claim 10, wherein the lubricant is selected from the group comprising magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate or combinations thereof.

22. The pharmaceutical composition of claim 10, wherein the lubricant is present in an amount from about 0.1 to about 8 percent wt. per total weight of the pharmaceutical composition.

23. The pharmaceutical composition of claim 10, wherein the compactable excipient is microcrystalline cellulose, calcium dibasic phosphate or a combination thereof.

24. The pharmaceutical composition of claim 10, wherein the pharmaceutical composition contains at least two extra-particular hydrophobic excipients.

25. The pharmaceutical composition of claim 10, wherein the hydrophobic release controlling agent(s) is present in an amount from about 1 to about 10 percent wt. per total weight of the pharmaceutical composition.

26. The pharmaceutical composition of claim 10, wherein the hydrophobic release controlling agent is selected from the group comprising hydrogenated vegetable oil, hydrogenated castor oil, ethyl cellulose or a combination thereof.

27. The pharmaceutical composition of claims 26, wherein hydrogenated castor oil is present in an amount from about 4 to about 20 percent wt. per total weight of the pharmaceutical composition.

28. The pharmaceutical composition of claim 1, wherein the pharmaceutical composition releases the levetiracetam contained therein from about 4 to about 16 hours after introduction of the dosage form into the dissolution medium when tested in 900 ml water at 37° C., 100 rpm using a USP dissolution test apparatus II (paddle).

29. The pharmaceutical composition of claim 1, wherein the controlled release layer is free or substantially free of hydrophilic pore-forming ingredients aside from plasticizer(s).

30. The pharmaceutical composition of claim 3, wherein the levetiracetam has a median particle size of from about 200 to about 1000 microns.

31. The pharmaceutical composition of claims 3, wherein the levetiracetam particles are not micronized.

32. A controlled release multiparticulate single dosage unit pharmaceutical composition comprising granules of levetiracetam, the pharmaceutical composition comprises:
f) levetiracetam is present in an amount of from about 50 to about 80 percent wt. per total weight of the pharmaceutical composition,
g) at least one hydrophobic polymer is present in an amount of from about 10 to about 20 percent wt. per total weight of the pharmaceutical composition,
h) at least one hydrophobic release controlling agent, as an extra-granular excipient, is present in an amount of from about 1 to about 10 percent wt. per total weight of the pharmaceutical composition,
i) at least one hydrophobic plasticizer, preferably is present in an amount of from about 1 to about 4 percent wt. per total weight of the pharmaceutical composition; and
j) at least one compactable excipient, as an extra-granular excipient, preferably is present in an amount of about 5 to about 20 percent wt. per total weight of the pharmaceutical composition,
wherein the granules of levetiracetam are coated with a controlled release layer.

33. The pharmaceutical composition of claim 32, wherein the granules of levetiracetam are compressed to a tablet.

34. The pharmaceutical composition of claim 1, comprising levetiracetam, polyvinylpyrrolidone, ethylcellulose, dibutyl sebacate, microcrystalline cellulose, hydrogenated castor oil, and magnesium stearate.

35. The pharmaceutical composition of claim 1, wherein pharmaceutical composition has stable dissolution profile when tested using 900 ml water at 37° C. and 100 rpm using a USP dissolution test apparatus II (Paddle) or using 900 ml buffer phosphate pH 6.0 at 37° C. using USP dissolution test apparatus I (Basket).

36. A method for preparing a controlled release multiparticulate pharmaceutical composition comprising controlled release levetiracetam particles comprising:
f) forming granules which comprise of levetiracetam and, optionally, one or more pharmaceutically acceptable excipients,
g) coating the granules with a controlled release layer,
h) optionally, blending the coated granules with one or more further pharmaceutically acceptable excipients,
i) compressing the resulting granules to form a tablet; and
j) optionally, coating the compressed tablet with a cosmetic non functional coat.

37. A method for preparing a controlled release multiparticulates pharmaceutical composition of levetiracetam comprising:
e) coating levetiracetam particles with a control release layer,
f) optionally, blending the coated particles with one or more pharmaceutically acceptable excipients,
g) compressing the resulting particles/blend to form a tablet; and
h) optionally, coating the compressed tablet with a cosmetic coat.

38. A pharmaceutical composition produced by the method of claims 36 or 37.

39. The controlled release multiparticulate pharmaceutical composition of claim 1, wherein the controlled release profile is up to about 50% release of levetiracetam in one hour, up to about 70% in two hours. and about 80% to about 100% in about 8 hours.
